(19) United States
(12) Patent Application Publication
(10) Pub. No.: US 2006/0275173 A1
(43) Pub. Date: Dec. 7, 2006

(54) Method for cleaning lipid deposits on silicone hydrogel contact lenses

(75) Inventors: Roya Borazjani, Fairport, NY (US); Joseph C. Salamone, Boca Raton, FL (US); Vicki Barniak, Fairport, NY (US); Robert Manchester, Lima, NY (US); David McCanna, Pittsford, NY (US)

Correspondence Address: Bausch & Lomb Incorporated, One Bausch & Lomb Place, Rochester, NY, 14604-2701, US

(73) Assignee: Bausch & Lomb Incorporated

(21) Appl. No.: 11/442,793

(22) Filed: May 30, 2006

Related U.S. Application Data
(60) Provisional application No. 60/687,086, filed on Jun. 3, 2005.

(51) Int. Cl. A61L 2/18

(52) U.S. Cl. 422/28; 424/70.31

(57) ABSTRACT

This invention is directed to a method of treating a silicone hydrogel lens to cleaning lipid deposits thereon. The method comprises administering to the contact lens a solution comprising a first straight chain polyether surfactant. The first straight chain polyether surfactant has an HLB value that is a minimum of about 18. The second straight chain polyether surfactant has an HLB value that is a minimum of about 12 and a maximum of about 18. The second surfactant is present in an amount effective to improve the ability to remove lipids from a silicone hydrogel lens.

### CROSS REFERENCE

This application claims the benefit of Provisional Patent Application No. 60/687,086 filed Jun. 3, 2005 and is incorporated herein by reference.

### FIELD OF INVENTION

This invention relates to an aqueous composition and methods for cleaning lipid deposits on medical devices, particularly, for contact lenses.

### BACKGROUND

Contact lenses are used by an increasing number of people as means of correcting vision and/or compensating for eye abnormalities. Worldwide, about 100 million people use contact lenses. In the U.S. alone, 34 million people wear contact lenses (http://www.medicalpost.com/mpcontent/article.jsp?content=/content/EXTRACT/RAW ART/3836/02B.html). However, contact lenses must usually be inserted and removed daily with scrupulous cleaning and disinfection between each wearing.

During wear and normal handling of contact lenses, microorganisms as well as biomolecules such as lipids, proteins, etc., can adhere to the contact lenses and contaminate the storage containers/solution. Furthermore, a tear film that contains proteins, lipids, and even microorganisms covers the surface of the eye. Any of these components found in the tear film, on the external surface of the eye or the surrounding skin, can be carried into the storage containers/solution for the contact lens. Then, the microorganisms that multiplied in the storage containers/solution can transfer to the eyes via contact lenses and become the pathogen that may cause eye infection resulting in impaired vision and blindness. Various solutions have been developed to clean these deposits and disinfect the microorganisms.

A “daily cleaner” comprised with various kinds of surfactants and disinfectants is recommended for daily use to remove most deposits and debris on contact lenses. In an approach to prevent protein deposits, contact lens solutions containing chemical agents such as cationic polymers were developed to prevent proteins from adhering to the contact lens surface of rigid gas permeable (RGP) and soft contacts lenses.

Solutions that wet the lenses before insertion in the eye are required for both the hard and soft types of contact lenses, although their formulations have tended to differ based on their different properties. After the contact lenses are inserted in the eye, ophthalmic solutions for rewetting, lubricating, and/or enhancing the comfort of the contact lens wearer are sometimes applied to the eye by means of a drop dispenser. Hypotonic and isotonic solutions for improving the comfort of wearing soft contact lenses by being added directly to the contact lens in the eye typically contain viscosity enhancing agents, lubricants, surfactants, buffers, preservatives, and salts.

Multipurpose solutions are popular because of the convenience of a single solution for cleaning, disinfecting and conditioning contact lenses immediately prior to insertion of the lens in the eye. Multipurpose solutions are also designed for use as a wetting agent, without rinsing, meaning that the solution must be ophthalmically safe for eye contact. This limits, to some extent, the type and concentration of both cleaning agents and biocides that can be employed in the solution as a preservative or disinfectant tends to be irritating to the eye. Additionally, the surface active agents must not inhibit the wetting or conditioning function of the solution.

Silicone hydrogel lenses have been difficult to clean and condition due to their tendency to absorb lipids into the material. Consequently, silicone hydrogels are suitable for daily use and are not recommended for extended wear.

U.S. Pat. No. 4,820,352 (Riedhammer et al.) discloses compositions for cleaning and conditioning contact lenses, where the primary cleaning agent is a specific class of polyethyleneoxy-polypropyleneoxy block copolymer adduct of ethylene diamine (also known as poloxamine). This patent describes compositions which are sufficiently nonirritating that a contact lens treated with the solution can be inserted directly in the eye.

U.S. Pat. No. 5,209,865 (Winterton et al.) discloses a conditioning solution for contact lenses that comprises a combination of a poloxamine and a poloxamer surfactant each having an HLB (hydrophilic-lipophilic balance) of seven or below. This patent describes a solution forming a uniform hydrophilic film on a lens surface for which proteins have very little affinity for a prophylactic effect to the lens.

U.S. Pat. No. 5,604,189 and U.S. Pat. No. 5,773,396 (Zhang et al.) disclose a composition for cleaning and wetting contact lenses comprising (i) a non-amine polyethyleneoxy-containing compound having an HLB of at least about 18, (ii) a surface active agent having cleaning activity for contact lens deposits that may have an HLB less than 18, and (iii) a wetting agent. An ethoxylated glucose derivative such as glucam can be employed as the wetting agent, also disclosed in U.S. Pat. No. 5,401,327 to Ellis et al. In another approach, Tyloxapol is employed as a conventional surface active agent in a multipurpose solution, which agent has cleaning activity for contact-lens deposits and has an HLB less than 18.

There has been constant need for ophthalmic solutions with better lipid cleaning properties for use with silicone hydrogel lenses. The deposits from cellular debris, protein and lipid accumulated over time can adsorb to the surface of contact lenses and irritate the eyes. Particularly, lipid deposits can reduce the transparency and impair the performance of the contact lenses. Lipid deposits on silicone hydrogel lenses during wear generate a hydrophobic surface that is not easily rewetted with currently used types and concentrations of surfactants in the lens care compositions. Most importantly, due to the nature of the hydrogel lens and its interaction with lipids, the duration of time that a silicone hydrogel can be used on a patient is limited. Cleaning techniques associated with RGP lenses typically cannot be used with silicone hydrogel lenses.

There is, therefore, a need for a composition with improved lipid cleaning properties while maintaining or increasing the biocidal efficacy of the product without adversely affecting comfort or safety in terms of the level of toxicity to eye tissue. It would also be desirable to have a composition that can be utilized as an eye drop, an eyewash solution, a contact lens care solution, or cleaning solution, a storing solution, a disinfectant, a cleaning-storing solution, and a cleaning disinfecting-storing solution. The invention addresses one or more of these and/or other needs.

### SUMMARY OF INVENTION

The present invention is a method of treating a silicone hydrogel lens to clean lipid deposits thereon, the method comprising administering to the silicone hydrogel contact lens a solution comprising a first straight chain polyether surfactant having an HLB value that is a minimum of about 18 and a second straight chain polyether surfactant having an HLB value that is a minimum of about 12 and a maximum of about 18. The surfactant (b) is present in an amount effective to improve the ability to remove lipids from a silicone hydrogel lens.

In one embodiment, the combined amount of surfactants (a) and (b) is a minimum of about 2.5 wt. %, about 3.0 wt. %, about 3.5 wt. %, about 4.0 wt. % or about 4.5 wt. % and a maximum of about 7.0 wt. %, 6.5 wt. %, 6.0 wt. %, 5.5 wt. % of the solution. Typically, the combined amount of surfactants (a) and (b) is about 5.0 wt. % of the solution.

In an embodiment, the first straight chained polyether surfactant has an HLB value that is a minimum of about 19, about 20, about 21 or about 22. Typically, the first straight-chained polyether surfactant has an HLB value that is about 22. Typically, the first straight chain polyether surfactant is present in a minimum amount of about 0.01 wt. %; about 0.05 wt. %; about 1 wt. %, about 1.5 wt. %, about 2 wt. %, about 2.5 wt. %, about 3 wt. % or about 3.5 wt. % and a maximum amount of about 7 wt. %, about 6 wt. %, about 5 wt. %, about 4.5 wt. %, about 4 wt. %, about 3.5 wt. % or about 3 wt. %.

In another embodiment, the first straight chain polyether surfactant is selected from the group consisting of Pluronic L35™, Pluronic F38™, Pluronic F68™, Pluronic 68LF™, Pluronic F77™, Pluronic F87™, Pluronic F88™, Pluronic F98™, Pluronic F108™, and Pluronic F127™. In yet another embodiment, the first straight chain polyether surfactant is F127™.

In another embodiment, the second straight chain polyether surfactant has an HLB value ranging is a minimum of about 12, about 13 about 14, or about 15 and a maximum of about 18, about 17, about 16, about 15. Typically, the second straight chain polyether surfactant has an HLB of about 15. In another embodiment, the straight chain polyether surfactant is selected from the group consisting of Pluronic L10™ (BASF); Pluronic L43™ (BASF); Pluronic L64™ (BASF); Pluronic P84™ (BASF); Pluronic P104™ (BASF) and Pluronic P105™ (BASF).

In another embodiment, the second straight chain polyether surfactant is present in a minimum amount of about 0.01 wt. %, about 0.05 wt. %, about 0.1 wt. %, about 0.5 wt. %, about 0.7 wt. % about 1 wt. % and a maximum amount of about 2 wt. %, about 1.5 wt. %, about 1.2 wt. %, about 1.0 wt. %, about 0.8 wt. %, about 0.7 wt. %, about 0.5 wt. %. Typically, the second straight chain polyether surfactant is present in an amount of about 0.1 wt. %.

In one embodiment, the composition or solution further comprises at least one member selected from the group consisting of buffering agents, a chelating agent, and an osmolality adjusting agent.

In another embodiment the composition or solutions further comprises one or more antimicrobial agents present in an amount effective to disinfect a medical device or preserve a composition or solution.

In still another embodiment, the composition further comprises a chelating agent and a buffering agent selected from the group consisting borate buffers, phosphate buffers, citrate buffers, aminoalcohol buffers, and good buffers.

In still another embodiment, the buffer agents are selected from the group consisting of borate buffers, phosphate buffers, citrate buffers, aminoalcohol buffers, good buffers, and mixtures thereof to maintain a pH that is a minimum of about 6, about 6.2, about 6, 5 about 7.0 and a maximum of about 8, about 7.8, about 7.5, about 7.2, about 7.0. Typically, the buffer agents have a pH of about 7.0

In one embodiment, the osmolality adjusting agent is present in concentration sufficient to provide solution osmolality that is a minimum of about 200 mOsm/kg, about 240 mOsm/kg, about 280 mOsm/kg, about 300 mOsm/kg, about 320 mOsm/kg, about 340 mOsm/kg, and a maximum that is about 400 mOsm/kg, about 380 mOsm/kg, about 360 mOsm/kg, about 340 mOsm/kg, about 320 mOsm/kg, about 300 mOsm/kg. Typically, the solution has an osmolality that is about 240-280 mOsm/kg.

In another embodiment, the composition or solution is used in an eye drop, or a contact lens care solution.

The invention also provides methods of cleaning lipids on a medical device with the aqueous composition of the invention. According to a preferred embodiment, the composition is sufficiently nonirritating that the aqueous composition can be administered directly in the eye for use as eye drops, or as a lens care solution. In another preferred embodiment, contact lenses, prior to placement in the eye, are soaked in an aqueous composition of multipurpose solution for cleaning lipid deposits.

The objects, features, and advantages of the various embodiments of the present invention will become more readily apparent from the following detailed description.

### DETAILED DESCRIPTION OF THE INVENTION

This invention is directed to aqueous compositions for cleaning lipid deposits on medical device, especially on contact lenses, and methods of using these compositions. The term “cleaning lipid deposits” includes preventing, removing, and/or reducing the formation of lipid deposits. Combinations according to the invention have been found to improve the lipid cleaning properties for contact lenses and prevent the overgrowth of harmful bacteria and molds without adversely affecting the comfort or safety in terms of the level of toxicity to eye tissue.

It has also been found that a combination of straight chain polyether surfactants, in particular poloxamers, is not only effective in cleaning lipid deposits, but is comfortable for use in the eye. The aqueous composition of the invention is believed to reduce the hydrophobicity of the lens surface of a silicone hydrogel, which may reduce the affinity of lipids to the surface of the silicone hydrogel lens surface. The combination of straight chain polyether surfactants according to the invention may not only prevent the deposition of lipids, but also to some extent may loosen deposits on the lens, wherein removal is assisted by the natural cleaning action of blinking. The silicone hydrogel lenses are soft contact lenses that contain siloxane-containing materials and have a high D_{K}. In one embodiment, the Dₖ of a silicone hydrogel lens is a minimum of about 100, about 130, about 140, about 160, about 180, about 200 or about 220.

The aqueous compositions or solutions that are used in the method of the present invention for cleaning lipid deposits on a silicone hydrogel medical device advantageously contain beneficial compositions of surfactants which yield highly effective lipid cleaning. Various other surfactants suitable for use in the invention are disclosed in McCutcheon's Detergents and Emulsifiers, North American Edition, McCutcheon Division, MC Publishing Co., Glen Rock, N.J. 07452 and the CTFA International Cosmetic Ingredient Handbook, Published by The Cosmetic, Toiletry, and Fragrance Association, Washington, D.C.

According to the invention, an aqueous composition for cleaning lipid deposits on a medical device comprising:
(a) a first straight chain polyether surfactant having an HLB value that is a minimum of about 18; and
(b) a second straight chain polyether surfactant having an HLB value that is a minimum of about 12 and a maximum of about 18,
wherein the second surfactant is present in an amount effective to improve the lipid cleaning effect of the composition.

Typically, the first straight chain polyether surfactant is present in a minimum amount of about 0.01 wt. %; about 0.05 wt. %; about 1 wt. %, about 1.5 wt. %, about 2 wt. %, about 2.5 wt. %, about 3 wt. % or about 3.5 wt. % and a maximum amount of about 7 wt. %, about 6 wt. %, about 5 wt. %, about 4.5 wt. %, about 4 wt. %, about 3.5 wt. % or about 3 wt. % of the solution or composition. Typically, the first straight chain polyether surfactant is present in an amount of about 4.5 wt. % of the solution or composition.

In another embodiment, the second straight chain polyether surfactant is present in a minimum amount of about 0.01 wt. %, about 0.05 wt. %, about 0.1 wt. %, about 0.5 wt. %, about 0.7 wt. % about 1 wt. % and a maximum amount of about 2 wt. %, about 1.5 wt. %, about 1.2 wt. %, about 1.0 wt. %, about 0.8 wt. %, about 0.7 wt. %, about 0.5 wt. %. Typically, the second straight chain polyether surfactant is present in an amount of about 0.1 wt. %.

The first and second straight chain polyether surfactants in the aqueous compositions of the invention comprise one or more chains or polymeric components having oxyalkylene (—O—R—) repeats units wherein R has 2 to 6 carbon atoms. Representative, first and second straight chain polyether surfactants comprise block polymers of two or more different kinds of oxyalkylene repeat units, the ratio of which determining the HLB of the surfactant. The first and second straight chain polyether surfactants are available from BASF Wyandotte Corp., Wyandotte, Mich., under the registered trademark “Pluronic™ (BASF).” For convenience purposes, the first and second straight chain surfactants employed in the aqueous composition disclosed herein will be referred to as Pluronic generally, and with a numerical suffix to identify a particular grade of material.

Pluronic are block copolymers consisting of propylene oxide (PO) and ethylene oxide (EO) blocks—specifically, they are poly(a-oxyethylene-b-oxypropylene-a-oxyethylene) triblock copolymers. Their solubility in water is generally good, but the properties of the individual block copolymers vary substantially. The nomenclature used for the block copolymers, and generally herein, is such that the first two figures, when multiplied by 100, represent the average molecular weight of the PO block, whilst the last figure, when multiplied by 10, represents the ethylene oxide content (% w/w) of the poloxamer. Thus, for Pluronic F127, the average molecular weight of the PO block is about 12000 Daltons with about 70% w/w/ethylene oxide content.

Grades of Pluronic surfactants available with molecular weights within a range having a minimum of about 1650 and a maximum of about 27,000. Properties of each grade within the series vary depending on the percent of hydrophilic units poly(oxyethylene) and molecular weight of hydrophobic units poly(oxypropylene) in the adduct. While all members within the series exhibit wetting and detergency properties, it was discovered that only certain members are suitable for use in the cleaning and conditioning solutions disclosed herein, due to the wide variation in performance characteristics regulated by their hydrophilic-hydrophobic balance. The Pluronic surfactants found suitable are those capable of demonstrating maximum cleaning efficiency in dispersing both protein and lipid deposits at ambient and elevated temperatures at lowest solution concentration without trade-offs in lens compatibility and toxicity levels, i.e. maintaining the lowest potential as an irritant to eye tissues.

In an embodiment, the first straight-chained polyether surfactant has an HLB value that is a minimum of about 19, about 20, about 21 or about 22. Typically, the first straight-chained polyether surfactant has an HLB value that is about 22. Suitable straight chain polyether surfactants having HLB value greater than or equal to about 18, (a) of the aqueous composition of the invention, include for example but are not limited to Pluronic F38™ (BASF) having a HLB of 31 and average molecular weight of 4700; Pluronic F68™ (BASF) having a HLB of 29 and an average molecular weight of 8400; Pluronic 68LF™ (BASF) having a HLB of 26 and an average molecular weight of 7700; Pluronic F77™ (BASF) having a HLB of 25 and an average molecular weight of 6600; Pluronic F87™ (BASF) having a HLB of 24 and an average molecular weight of 7700; Pluronic F88™ (BASF) having a HLB of 28 and an average molecular weight of 11400; Pluronic F98™ (BASF) having a HLB of 28 and an average molecular weight of 13000; Pluronic F108™ (BASF) having a HLB of 27 and an average molecular weight of 14600; Pluronic F127™ (BASF) having a HLB of 22 and an average molecular weight of 12600; Pluronic L35™ (BASF) having a HLB of 19 and an average molecular weight of 1900.

Suitable straight chain polyether surfactants having HLB having a minimum of about 12 and a maximum of about 18 of the aqueous compositions of the invention, include for example but are not limited to the following:
Pluronic L10™ (BASF) having a HLB of 14 and average molecular weight of 3200;
Pluronic L43™ (BASF) having a HLB of 12 and average molecular weight of 1850;
Pluronic L64™ (BASF) having a HLB of 15 and average molecular weight of 2900;
Pluronic P84™ (BASF) having a HLB of 14 and average molecular weight of 4200;
Pluronic P104™ (BASF) having a HLB of 13 and average molecular weight of 5900;
Pluronic P184™ (BASF) having a HLB of 15 and average molecular weight of 6500;

A particularly preferred Pluronic surfactant of this group is Pluronic P105™.

Most preferred Pluronic surfactants are a combination of Pluronic P123™ and Pluronic P105™.

The HLB of a surfactant is an important factor in determining the emulsification characteristics of a polyether surfactant. In general, surfactants with lower HLB values are more lipophilic, while surfactants with higher HLB values are more hydrophilic. The HLB values of various poloxamines and poloxamers are provided by BASF Wyandotte Corp., Wyandotte, Mich.

Relatively high HLB values greater than about 18, or even more preferably 22 or higher, indicate a lower affinity for both hydrophobic molecules and/or surfaces, such as lipids and hydrophilic molecules from silicone hydrogel contact lenses.

The straight poly(ethylene oxide-propylene oxide-ethylene oxide) (PEO-PPO-PEO) block copolymers that are a minimum of 12 and a maximum of 18 or below, Pluronic, is present in an amount effective to improve the lipid cleaning effect of the composition. This combination of different HLB Pluronics set for the in this patent demonstrates an unexpected, enhanced cleaning lipid properties for silicone hydrogel contact lenses.

Such polyether surfactants, the first and second straight chain polyether surfactants of the aqueous compositions, are preferably employed in the invention in total combined amount that is a minimum of about 2.5 wt. %, about 3.0 wt. %, about 3.5 wt. %, about 4.0 wt. % or about 4.5 wt. % and a maximum of about 7.0 wt. %, 6.5 wt. %, 6.0 wt. %, 5.5 wt. % of the solution. Typically, the combined amount of surfactants (a) and (b) is about 5.0 wt. % of the solution.

The aqueous composition according to the invention are physiologically compatible. Specifically, the solution must be “ophthalmically safe” for use with a contact lens, meaning that a contact lens treated with the solution is generally suitable and safe for direct placement on the eye, that is, the solution is safe and comfortable for daily contact with the eye via a contact lens that has been wetted with the solution. An ophthalmically safe solution has a tonicity and pH that is compatible with the eye and comprises materials, and amounts thereof, that are non-cytotoxic according to ISO (International Standards Organization) standards and U.S. FDA (Food & Drug Administration) regulations. The solution should be sterile in that the absence of microbial contaminants in the product prior to release must be statistically demonstrated to the degree necessary for such products.

An aqueous composition of the invention can be applied in the form of an eye drop, or a contact lens care solution. The eye drop solution can be selected from the group consisting of a solution to soothe eye irritation, a moisturizing solution, a contact lens rewetting solution, and a contact lens lubricating solution. The contact lens care solution can be selected from the group consisting of a cleaning solution, a storing solution, a disinfecting solution, a conditioning solution, a wetting solution and a multipurpose solution.

According to various preferred embodiments of the invention, the compositions are likewise suitable for disinfecting a contact lens soaked therein. In addition to water, it is preferred that the compositions also include at least one antimicrobial agent, especially a non-oxidative antimicrobial agent that derives its antimicrobial activity through a chemical or physicochemical interaction with organisms. So that the contact lenses treated with the composition may be instilled directly in the eye, i.e., without rinsing the contact lens with a separate composition, the antimicrobial agent needs to be an ophthalmically acceptable antimicrobial agent.

Suitable antimicrobial agents for use in the invention include quaternary ammonium salts. Suitable quaternary ammonium salts for use in the invention include for example but are not limited to poly[(dimethyliminio)-2-butene-1,4-diyl chloride] and [4-tris(2-hydroxyethyl)ammonio]-2-butenyl-ω-[tris(2-hydroxyethyl)ammonio]dichloride (Chemical Abstracts Registry Number 75345-27-6) generally available as Polyquaternium-1 from Onyx Corporation. Also suitable are biguanides and their salts, such as 1,1′-hexamethylene-bis[5-(2-ethylhexyl)biguanide] (Alexidine) and poly(hexamethylene biguanide) (PHMB) available from ICI Americas, Inc., Wilmington Del. under the trade name Cosmocil CQ, benzalkonium chloride (BAK) and sorbic acid.

One or more antimicrobial agents are present in the compositions in an amount effective for disinfecting a contact lens, as found in conventional lens soaking and disinfecting solutions. Preferably, the antimicrobial agent will be used in a disinfecting amount or an amount that is a minimum of about 0.0001 wt. %, 0.0005 wt. %, 0.001 wt. %, 0.005 wt. %, 0.01 wt. %, 0.05 wt. % and a maximum of about 0.5 wt. %, 0.1 wt. %, 0.005 wt. %, 0.001 wt. % of the solution or composition. A disinfecting amount of an antimicrobial agent is an amount that will at least partially reduce the microorganism population in the formulations employed.

Contact lens care solutions require disinfection and or preservative compliance with FDA (510 (k)) Guidance Document for contact lens products. These procedures measure the extent of viability loss of representative microorganisms at established time intervals.

FDA (510 (k)) Guidance Document's recommended test organisms for both disinfecting stand-alone and preservative efficacy testing are composed of three bacteria (Pseudomonas aeruginosa ATCC 9027, Stapylococcus aureus ATCC 6538, and Serratia marcescens ATCC 13880) and two fungi (Candida albicans ATCC 10231, and Fusarium solani ATCC 36031). The performance requirement biocidal stand alone testing calls for a 3 log reduction for bacterial cells and a one log reduction for each fungi. At day 28, after a rechallenge on day 14, the performance requirement for preservative efficacy testing calls for a reduction of 3.0 logs per bacteria and ±0.5 for fungi.

Aqueous compositions of the invention may also contain various other components including for example but not limited to one or more chelating and/or sequestering agents, one or more osmolality adjusting agents, one or more surfactants, one or more buffering agents and/or one or more wetting agents. Chelating agents, also referred to as sequestering agents, are frequently employed in conjunction with an antimicrobial agent. These agents bind heavy metal ions, which might otherwise react with the lens and/or protein deposits and collect on the lens. Chelating agents are well known in the art, and examples of preferred chelating agents include ethylenediaminetetraacetic acid (EDTA) and its salts, especially disodium EDTA. Such agents are normally employed in an amounts that is a minimum of about 0.01 wt. %, 0.03 wt. %, 0.05 wt. % and/or a maximum of about 2.0 wt. %, about 0.8 wt. %, about 0.5 wt. % or about 0.3 wt. % of the total solution. Other suitable sequestering agents include gluconic acid, citric acid, tartaric acid and their salts, e.g., sodium salts. Aqueous compositions of the invention may be designed for a variety of osmolalities, but it is preferred that the compositions range from hypotonic to isotonic with respect to eye fluids.

In one embodiment, the osmolality adjusting agent is present in concentration sufficient to provide solution osmolality that is a minimum of about 200 mOsm/kg, about 240 mOsm/kg, about 280 mOsm/kg, about 300 mOsm/kg, about 320 mOsm/kg, about 340 mOsm/kg, and a maximum that is about 400 mOsm/kg, about 380 mOsm/kg, about 360 mOsm/kg, about 340 mOsm/kg, about 320 mOsm/kg, about 300 mOsm/kg. Typically the osmolality is a minimum of about 220 and a maximum of about 310 mOsm/Kg—preferably about 280 mOsm/Kg. One or more osmolality adjusting agents may be employed in the composition to obtain the desired final osmolality. Examples of suitable osmolality adjusting agents include, but are not limited to sodium and potassium chloride, monosaccharides such as dextrose, calcium and magnesium chloride, and low molecular weight polyols such as glycerin and propylene glycol. Typically, these agents are used individually in amounts that are a minimum of about 0.01 wt. % and a maximum of about 5 wt. %.

Aqueous compositions of the invention have an ophthalmically compatible pH. In still another embodiment, the buffer agents maintain a pH that is a minimum of about 6, about 6.2, about 6, 5 about 7.0 and a maximum of about 8, about 7.8, about 7.5, about 7.2, about 7.0. Typically, the buffer agents have a pH of about 7.0.

One or more conventional buffers may be employed to obtain the desired pH value. Suitable buffers include for example but are not limited to borate buffers based on boric acid and/or sodium borate, phosphate buffers based on Na₂HPO₄, NaH₂PO₄ and/or KH₂PO₄, citrate buffers based on sodium or potassium citrate and/or citric acid, sodium bicarbonate, aminoalcohol buffers, Good buffers and combinations thereof. Generally, buffers will be used in amounts that are a minimum of about 0.05 wt. %, 0.1 wt. %, 0.3 wt. %, 0.6 wt % and a maximum of about 2.5 wt. %, 2.0 wt. %, 1.5 wt. %, or 1.0. wt. % of the solution or composition

Aqueous compositions may likewise include a wetting agent, to facilitate the composition wetting the surface of a contact lens. Within the art, the term “humectant” is also commonly used to describe these materials. A first class of wetting agents are polymer wetting agents. Examples of suitable wetting agents include for example but are not limited to poly(vinyl alcohol) (PVA), poly(N-vinylpyrrolidone) (PVP), cellulose derivatives and poly(ethylene glycol). Cellulose derivatives and PVA may be used to also increase viscosity of the composition, and offer this advantage if desired. Specific cellulose derivatives include for example but are not limited to hydroxypropylmethylcellulose, carboxymethylcellulose, methylcellulose, hydroxyethylcellulose, and cationic cellulose derivatives. As disclosed in U.S. Pat. No. 6,274,133, cationic cellulosic polymers also help prevent accumulation of lipids and proteins on a hydrophilic lens surface. Such cationic cellulosic polymers include for example but are not limited to water soluble polymers commercially available under the CTFA (Cosmetic, Toiletry, and Fragrance Association) designation Polyquaternium-10, including the cationic cellulosic polymers available under the trade name UCARE® Polymers from Amerchol Corp., Edison, N.J., such as for example but not limited to Polymer JR™. Generally, these cationic cellulose polymers contain quaternized N,N-dimethylamino groups along the cellulosic polymer chain.

Another suitable class of wetting agents is non-polymeric wetting agents. Examples may include glycerin, propylene glycol, and other non-polymeric diols and glycols. The specific quantities of wetting agents used in the invention will vary depending upon the application. However, the wetting agents will typically be included in an amount that is a minimum of about 0.01 wt. %, 0.05 wt. %, 0.1 wt. % or 0.5 wt. % and a maximum of about 5 wt. %, about 3 wt. %, about 2.0 wt. %, about 1.5 wt. % of the solution or composition.

It will be understood that some constituents possess more than one functional attribute. For example, cellulose derivatives are suitable polymeric wetting agents, but are also referred to as “viscosity increasing agents” to increase viscosity of the composition if desired. Glycerin is a suitable non-polymeric wetting agent but is also may contribute to adjusting tonicity.

Aqueous compositions of the invention can be utilized as an eye drop solution or contact lens care solution by optimizing the concentration of the disinfectant to biostatic agent in case of an eye-drop formula or biocidal for multipurpose solution. When used as an eye drop solution, the aqueous composition may soothe eye irritation act as a moisturizer, as a contact lens rewetting solution, or as a contact lens lubricating solution. The contact lens care solution is selected from the group consisting of a cleaning solution, a storing solution, a disinfecting solution, a conditioning solution, a wetting solution, or a multi-purpose solution. Preferably, aqueous compositions are applied in the form of drops to a contact lens while it is worn in the eye and which is useful for rewetting or lubricating the lens as well as for prophylactically cleaning the lens by preventing the deposition of lipids.

Such aqueous compositions can be used to prevent the overgrowth of harmful Gram-positive and Gram-negative bacteria such as Pseudomonas aeruginosa, Serratia marcescens and Staphyllococcus aureus, as well as harmful molds on the lens surfaces during wear, or during the soak time, while being gentle and non-toxic against corneal epithelial cells.

The invention is especially useful for cleaning a contact lens while it is worn in the eye. Thus, as mentioned above, aqueous compositions according to the invention are especially advantageous with people who are prone to heavy lipid or like deposition or who wear lenses under an extended-wear, or continuous-wear regime. Extended wear is defined as a lens that is worn overnight, during sleep, preferably capable of wear for a week or more. Continuous wear is defined as a lens that is worn for at least 1 month.

The aqueous compositions of the invention are typically sold in a wide range of small volume containers from 1 to 30 ml in size, preferably 1 ml to 20 ml in size. Such containers can be made from HDPE (high density polyethylene), LDPE (low density polyethylene), polypropylene, poly(ethylene terepthalate) and the like. Flexible bottles having conventional drop dispensing tops are especially suitable for use with the present invention. Solutions according to the invention may suitably be applied as follows. During wear, about one or two drops are placed directly onto each lens whenever needed. Thereafter, the wearer should blink several times. It is also possible to use a spray mist to deliver the formulation to the eye.

The aqueous composition of the invention may be effectively used in cleaning lipid deposits on both hard and soft type contact lenses by any of the well-recognized methods. For example, when the wearer of contact lenses removes the lens from the eyes, the lens may be rubbed with the cleaning solution followed by “cold” soaking at room temperature for a period ranging from about four to twelve hours. The lenses are then removed from the solution and replaced on the eyes. The wearer may optionally rinse the lenses in a preserved saline solution before replacing the lenses on the eyes.

In addition to the cold soaking method, the solutions disclosed herein are adaptable for use in other type of equipment such as ultrasonic cleaners. Furthermore, because the solutions are also stable when heated to temperatures in the range of 80° to 90° C. They are also adaptable for use with high temperature disinfecting methods. Typically, lenses are heated to 80° C. in a disinfecting unit containing the cleaning and conditioning solution for a time period of at least 10 minutes, removed and rinsed with isotonic saline.

The following specific experiments and examples demonstrate the compositions and methods of the present invention. However, it is to be understood that these examples are for illustrative purposes only and do not purport to be wholly definitive as to conditions and scope. All percentages are by weight of the solution, unless indicated otherwise.

### EXAMPLES

In the examples below, certain chemical ingredients are identified by the following abbreviations.
HAP: HAP buffer, phosphate-buffered saline (PBS) with 0.5 U of aprotinin per ml-0.05% human serum albumin-3 mM D-glucose
Polymer JR®: cationic polysaccharide, polyquaternium-10
Alexidine 2HCl: quaternary ammonium salt, 1,1′-hexamethylene-bis[5-(2-ethylhexyl)biguanide]

### Example 1

### Making the Formulations

Table 1 lists the ingredients of the base formulation for the examples.

TABLE 1
Base Formulation
A. Base Formulation

| Ingredient | % W/W |
|---|---|
| Sodium Chloride | 0.047 |
| Boric Acid | 0.85 |
| Sodium Phosphate (Monobasic) | 0.15 |
| Sodium Phosphate (Dibasic) | 0.31 |
| HAP | 0.1 |
| Polymer JR | 0.02 |
| Pluronic & Tetronic Copolymers | See Individual Formulations |
| Alexidine 2HCL | 3.0 ppm |
| PH = 6.9-7.1 | |
| Osmo.(mOsmo/Kg) = 220-300 | |

Table 2 shows actual surfactant concentrations of Formulations I to IV. All formulations are prepared by combining the respective amounts of the base formulation, surfactants with water. The formulations are filtered thereafter.

TABLE 2
Compositions of Formulations I to IV

| Base | | | | | | | | |
|---|---|---|---|---|---|---|---|---|
| Formulation Plus | | | | | | | | |
| SURFAC- | | Formulation # | | | | | | |
| TANTS | HLB | I | II | III | IV | V | VI | VII |
| PLURONIC | 22 | 3% | 3% | 4.5% | 4.5% | 3% | 2.5% | |
| F127 | | | | | | | | |
| PLURONIC | 15 | | 0.1% | | 0.1% | 0.05% | 0.25% | |
| P105 | | | | | | | | |
| TETRONIC | 24 | 1.5% | 1.5% | | | 1.5% | 1.25% | |
| 1107 | | | | | | | | |

### Example 2

### Lipid Cleaning and Toxicity Studies

Lipid cleaning studies were done based on a spectrophotometric measurement of the suspension, which includes the mixture of an orange dye (Sudan I) with cholesterol. Ten ml volume of formulations was tested for their ability of dissolving the lipid for 24 hour in room temperature. The higher the absorbance values, the higher the lipid cleaning efficacy of the formulations. Addition of 0.1% of P105 (Formula IV: HLB value of 15) has a statistically increased the lipid cleaning values from the control composition (Formula III).

Toxicity data was generated using a cell culture model for predicting the ocular irritation potential of new contact lens care compositions (Na-Fluorescent permeability assay). All these formulations have shown permeability values below the level of control formulation I. The control formulation I is a currently marketed and safe ophthalmic care product.

TABLE 3
Formulations I to IV Lipid Cleaning and Toxicity

Formulation #

| | I | II | III | IV |
|---|---|---|---|---|
| Lipid Cleaning | 330 | 390 | 594 | 707 |
| Value | | | | |
| Toxicity | 49 | 35 | 45 | 48 |
| (Fluorescent Unit) | | | | |

### Example 3

### Preservative Efficacy

Table 4 shows the results of the effect of preservative efficacy of formulations I to IV. Preservative efficacy was tested according to the procedures disclosed above. Each of formulations I to IV passed the preservative efficacy test.

TABLE 4
Formulations I to IV Preservative Efficacy

| | | Formulation # | | | |
|---|---|---|---|---|---|
| PE Test Results | | I | II | III | IV |
| S. aureus | 14 day | Passed | Passed | Passed | Passed |
| | 28 day | Passed | Passed | Passed | Passed |
| P. aeruginosa | 14 day | Passed | Passed | Passed | Passed |
| | 28 day | Passed | Passed | Passed | Passed |
| E. coli | 14 day | Passed | Passed | Passed | Passed |
| | 28 day | Passed | Passed | Passed | Passed |
| C. albicans | 14 day | Passed | Passed | Passed | Passed |
| | 28 day | Passed | Passed | Passed | Passed |
| A. niger | 14 day | Passed | Passed | Passed | Passed |
| | 28 day | Passed | Passed | Passed | Passed |

### Example 4

### Dose Response for P105 in Pluronic F127 and Tetronics 1104

Formulations I, II, V and VI were prepared. The lipid cleaning test was performed according to the procedure of Example II for lipid cleaning. The higher the lipid cleaning value represents a higher surfactant activity. The results show that a significant increase in lipid cleaning of the control formulation (Formulation I) occurred when 0.05 wt. % to 0.1 wt. % of P105 is added.

TABLE 5

| | | Formulation # | | | |
|---|---|---|---|---|---|
| SURFACTANTS | HLB | I | II | V | VI |
| PLURONIC | 22 | 3% | 3% | 3% | 3% |
| F127 | | | | | |
| PLURONIC | 15 | | 0.1% | 0.05% | 0.01% |
| P105 | | | | | |
| TETRONIC | 24 | 1.5% | 1.5% | 1.5% | 1.5% |
| 1107 | | | | | |

| | | | | |
|---|---|---|---|---|
| Lipid Cleaning | 528 | 575 | 547 | 527 |
| Value | | | | |

These data show a dose response and threshold limit for P105 in a solution containing 3% Pluronics F127 and 1.5% Tetronics 1107.

TABLE 6

Formulation #

| SURFACTANTS | HLB | II | VII |
|---|---|---|---|
| PLURONIC | 22 | 3% | 2.5% |
| F127 | | | |
| PLURONIC | 15 | 0.1% | 0.25% |
| P105 | | | |
| TETRONIC | 24 | 1.5% | 1.25% |
| 1107 | | | |

| | | |
|---|---|---|
| Lipid Cleaning Value | 581 | 591 |

These data show that you can have a lower total surfactant concentration and maintain lipid cleaning with the use of increased concentration of P105.

What is claimed is:

1. A method of treating a silicone hydrogel lens to clean lipid deposits thereon, the method comprising administering to the contact lens a solution comprising:
(a) a first straight chain polyether surfactant having an HLB value that is a minimum of about 18; and
(b) a second straight chain polyether surfactant having an HLB value that is a minimum of about 12 and a maximum of about 18;
wherein the surfactant (b) is present in an amount effective to improve the ability to remove lipids from a silicone hydrogel lens.

2. The method of claim 1, wherein the combined amount of surfactants (a) and (b) is from a minimum of about 2.5 wt. % and a maximum of about 7.0 wt. %.

3. The method of claim 1, wherein the first straight chained polyether surfactant has an HLB value that is a minimum of about 22.

4. The method of claim 1, wherein the first straight chain polyether surfactant is selected from the group consisting of Pluronic L35™, Pluronic F38™, Pluronic F68™, Pluronic 68LF™, Pluronic F77™, Pluronic F87™, Pluronic F88™, Pluronic F98™, Pluronic F108™, and Pluronic F127™.

5. The method of claim 1, wherein the second straight chain polyether surfactant is selected from the group consisting of Pluronic L10™ (BASF); Pluronic L43™ (BASF); Pluronic L64™ (BASF); Pluronic P84™ (BASF); Pluronic P104™ (BASF) and Pluronic P105™ (BASF).

6. The method of claim 1, wherein the straight chain polyether surfactant (b) is present in an amount that is a minimum of about 0.01 wt. % and a maximum of about 2 wt. %.

7. The method of claim 1, wherein the composition further comprises at least one member selected from the group consisting of buffering agents, a chelating agent, and an osmolality adjusting agent.

8. The method of claim 1, wherein the composition further comprises one or more antimicrobial agents present in an amount effective to disinfect a medical device or preserve a solution.

9. The method of claim 7, wherein the composition further comprises a chelating agent and a buffering agent selected from the group consisting borate buffers, phosphate buffers, citrate buffers, aminoalcohol buffers, and good buffers.

10. The method of claim 9, wherein the buffer agents are selected from the group consisting of borate buffers, phosphate buffers, citrate buffers, aminoalcohol buffers, good buffers, and mixtures thereof to maintain a pH from about 6 to about 8.

11. The method of claim 7, wherein the osmolality adjusting agent is present in concentration sufficient to provide solution osmolality of from about 200 to about 400 mOsm/kg.

12. The method of claim 1, wherein the composition is used in an eye drop, or a contact lens care solution.
